(19) United States
(12) Patent Application Publication
(10) Pub. No.: US 2022/0087852 A1
(43) Pub. Date: Mar. 24, 2022

(54) STENT DEVICES AND METHODS TO INCREASE PATENCY OF A BODY OPENING

(71) Applicant: Boston Scientific Scimed, Inc., Maple Grove, MN (US)

(72) Inventors: Gary Gilmartin, Foxford (IE); Martyn G. Folan, Loughrea/Co. Galway (IE); Geraldine Alice Toner, Raphoe (IE); Michael Rodgers, Mayo (IE)

(21) Appl. No.: 17/474,651

(22) Filed: Sep. 14, 2021

Related U.S. Application Data
(60) Provisional application No. 63/082,517, filed on Sep. 24, 2020.

(51) Int. Cl. A61F 5/445

(52) U.S. Cl. CPC A61F 5/445

(57) ABSTRACT

A stent formed of a radially-outwardly expandable material to exert a radially expansive force to inhibit migration. The stent includes a retention member configured to retain the stent in place relative to the body lumen in which the stent is placed. The retention member may further contribute to inhibiting migration of the stent and/or tissue at which the stent is deployed. Various features may be provided on the retention member to adapt the stent for use with a stoma and/or ostomy appliances or other particular locations in the body benefiting from the stent.

### PRIORITY

The present application is a non-provisional of, and claims the benefit of priority under 35 U.S.C. § 119 to, U.S. Provisional Application Ser. No. 63/082,517, filed Sep. 24, 2020, the disclosure of which is hereby incorporated herein by reference in its entirety for all purposes.

### FIELD

The present disclosure relates generally to the field of implantable medical devices. In particular, the present disclosure relates to medical devices, systems, and methods which may be used for treatment of a body opening such as a stoma.

### BACKGROUND

Various body openings may exist or may be created which must be maintained in an open configuration despite various constricting forces thereon. For instance, a stoma is an artificial opening in the body created during ostomy surgery to treat a variety of diseases (e.g., bowel/bladder cancer, IBD, diverticulitis, fecal incontinence), such as to allow egress of materials from the body. There are several complications associated with stomas, which affect the long-term success of the procedure. Complications associated with the stomas include stenosed stomas (constriction of the stoma lumen), prolapsed stomas (moved or displaced from the proper position), and retracted stomas (receded stoma). Other types of body openings may present similar complications with regard to maintaining patency of the opening.

There is a need in the art for ways to maintain patency of body openings, such as a surgically created body opening, such as an opening in an exterior wall of a body. There is a further need to reduce the rate of stoma complications and/or improve patency of stomas.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

The present disclosure relates to a device that can be placed into a body opening, such as a stoma, particularly a newly formed stoma, such as to reduce the associated complication rates and/or to increase patency rate, and related systems and methods.

In various embodiments described or otherwise within the scope of the present disclosure, a stent is disclosed for deployment through a body opening and into a body lumen or passage or vessel (such terms being used interchangeably herein without intent to limit), and including a radially-expandable body portion movable between an unexpanded or delivery or constrained configuration (such terms being used interchangeably herein without intent to limit), with a first (unexpanded) diameter, and an expanded configuration with a second, expanded diameter larger than the first diameter and sized to exert a radially expansive force on the body lumen. The stent includes a retention member disposed on an end of the stent and configured to be placed external to the body lumen. In some embodiments, the retention member is sized and shaped and configured to surround and cover the body opening to the body lumen. In some embodiments, the retention member is sized and shaped and configured to retain the stent in place relative to the body lumen.

In the unexpanded configuration, the stent is sized to pass into a body lumen. The radially expansive force exerted by the radially-expandable stent wall on the lumen wall may be selected to inhibit migration of the stent and/or to maintain patency of the body lumen. In some embodiments, the stent exerts a radially expansive force on the vessel wall to retain the vessel wall in place relative to the opening in the body through which the stent is inserted into the vessel, such as to retain in place an external portion of a vessel wall extending through a stoma opening.

In some embodiments, the retention member has a diameter selected to be larger than the diameter of the stoma in which the stoma device is to be placed to contact an exterior body wall surrounding a stoma in which the stoma device is placed. Alternatively or additionally, the retention member distributes forces on the stoma device around the stoma to dissipate forces and to protect the stoma.

In some embodiments, the stoma device further includes a saddle region, the retention member extending radially-outwardly from the saddle region and shaped and configured to form a gap between a proximal side of the retention member and the stoma covered by the retention member. In some embodiments, the retention member has a distal side facing toward the saddle region and angled with respect to a longitudinal axis of the stoma device and toward the saddle region to form the gap. Alternatively or additionally, the stoma device further includes a spacer positioned along a distal side of the retention member facing the saddle region to form the gap. Alternatively or additionally, the stoma device further includes a covering member proximal to the retention member and sized to extend beyond the perimeter of the retention member. In some embodiments, the covering member is flexible and liftable away from the retention member to expose the stoma covered by the retention member.

In some embodiments, the stoma device further includes a connector extending from the retention member and configured for coupling with an ostomy appliance.

In some embodiments, the stoma device further includes an internal retention member configured to be positioned within the vessel wall internal to the stoma.

In various embodiments described or otherwise within the scope of the present disclosure, a stoma device is disclosed configured for deployment in a stoma. The stoma device includes a radially-expandable stent having a retention member disposed on an end thereof, the retention member configured to be placed external to the stoma and sized and shaped and configured to cover the stoma and to adapt the stoma device for use in the stoma.

In some embodiments, the retention member has a diameter selected to be larger than the diameter of the stoma in which the stoma device is to be placed and is sized and shaped and configured to contact an exterior body wall surrounding a stoma in which the stoma device is placed to distribute forces on the stoma device around the stoma to dissipate forces and to protect the stoma.

In some embodiments, the stoma device further includes a saddle region, the retention member extending radially-outwardly from the saddle region and shaped and configured to form a gap between a proximal side of the retention member and the stoma covered by the retention member. In some embodiments, the retention member has a distal side facing toward the saddle region and angled with respect to a longitudinal axis of the stoma device and toward the saddle region to form the gap. In some embodiments, the stoma device further includes a spacer positioned along a distal side of the retention member facing the saddle region to form the gap.

In some embodiments, the stoma device further includes a covering member proximal to the retention member and sized to extend beyond the perimeter of the retention member. In some embodiments, the covering member is flexible and liftable away from the retention member to expose the stoma covered by the retention member.

In some embodiments, the stoma device further includes a connector extending from the retention member and configured for coupling with an ostomy appliance.

In accordance with other aspects, a method of inhibiting migration of a stoma is disclosed, the method including: inserting a stoma device having a radially-expandable stent wall into the stoma, the radially-expandable stent wall movable between an unexpanded configuration, with a first diameter sized to pass into the stoma, and an expanded configuration with a second, expanded diameter larger than the first diameter and sized to support the stoma; and selecting the stoma device to exert a radially-expansive force on the stoma to retain the stoma device in place relative to the stoma to prevent migration of both the stoma device and the stoma.

In some embodiments, the stoma stent further includes a retention member configured to be placed external to the stoma, the method further including selecting a stoma stent with a retention member sized and shaped and configured to cover the stoma site, and covering the stoma with the retention member upon placement of the stoma stent within the stoma.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic illustration of a stoma formed in a human body.

FIG. 2 is a schematic cross-sectional view of a portion of a human body with a stent in accordance with principles of the present disclosure extending therethrough.

FIG. 3 is a schematic cross-sectional view of a portion of a human body with a stent in accordance with principles of the present disclosure extending therethrough.

FIG. 4A is a schematic cross-sectional view of a portion of a human body with a stent in accordance with principles of the present disclosure extending therethrough and showing another example of an embodiment of a retention member formed in accordance with principles of the present disclosure.

FIG. 4B is a side view similar to FIG. 4A, but with the retention member lifted away from the body wall.

FIG. 5 is a schematic cross-sectional view of a portion of a human body with another example of stent in accordance with principles of the present disclosure extending therethrough and showing another example of an embodiment of a retention member, with a collection connector, formed in accordance with principles of the present disclosure.

FIG. 6 is a perspective view of an example of an embodiment of a stent in accordance with principles of the present disclosure.

FIG. 7 is a schematic cross-sectional view of a portion of a human body with another example of stent in accordance with principles of the present disclosure extending therethrough.

FIG. 8 is a schematic cross-sectional view of a portion of a human body with another example of stent in accordance with principles of the present disclosure extending therethrough.

FIG. 9 is a view similar to that of FIG. 3 with a stoma stent similar to the stent of FIG. 3 but with a modified feature.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the medical professional (or clinician or technician or operator or physician, etc., such terms being used interchangeably without intent to limit or otherwise), such as when using a device (e.g., introducing the device into a patient, or during implantation, positioning, or delivery), and “distal” refers to the direction or location furthest from the medical professional, such as when using the device (e.g., introducing the device into a patient, or during implantation, positioning, or delivery). “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a strut, a channel, or a bore.

Various embodiments of stoma stents will now be described. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present invention is not limited to only the embodiments specifically described herein.

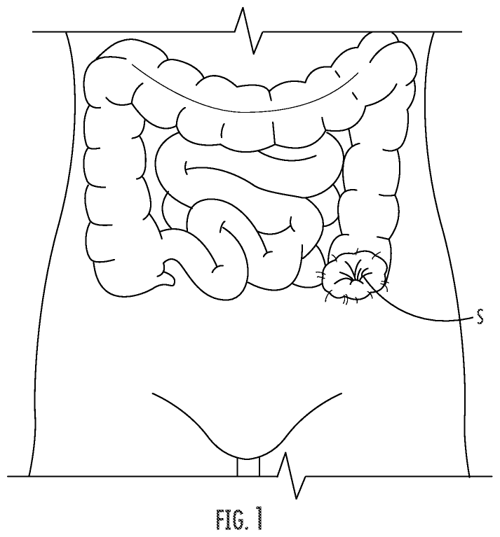

In accordance with various principles of the present disclosure, an expandable device such as a stent is used in a body opening, such as an artificial body opening such as a stoma with a stent body positioned at least partially within a body lumen. The device is described herein as being used, for example, in a colostomy (as illustrated in FIG. 1), ileostomy, or urostomy, or for fecal incontinence. However, other body openings may benefit from a stent as disclosed herein. The disclosed stent may have a flexible self-expanding (e.g., radially-expanding) stent body, such as formed of a shape memory material configured to be deployed in a body lumen or vessel. For example, the stent may be formed of multiple braided wires or a single braided wire. Braiding technology may be used to allow the stent to be delivered in a compact (e.g., constrained or unexpanded) configuration having a delivery diameter, and for at least a portion of the stent to expand, once delivered (such as in a stoma), to an expanded deployed diameter larger than the delivery diameter. Once the stent is deployed, the stent body, or a portion thereof, may expand under radially expansive forces which not only expand the stent body (or portion thereof) but also maintain patency of the lumen in which the stent body is positioned or the opening of the body opening through which the stent is deployed. For instance, when the stent is deployed in a stoma, the stent body is expanded within the stoma and the vessel wall extending to (and generally also through) the stoma opening so the stoma does not get occluded. In addition, in accordance with various principles of the present disclosure, embodiments of disclosed stents have a retention member shaped and configured to expand outside the lumen in which the stent body is positioned, and generally in a direction transverse to a longitudinal axis of such lumen. The retention member is shaped and configured and formed to have a sufficient holding or retention force to maintain the position of the stent with respect to the lumen and to reduce or inhibit or eliminate migration of the stent. The retention member may also be shaped and configured and formed to maintain the configuration of the opening region (e.g., inhibit or prevent prolapse or other undesired shifting of the opening or a vessel extending through the opening) that surrounds the lumen in which the stent body is positioned.

The radially expansive forces may be generated by the manner in which the stent is formed and/or the material from which the stent is formed. For instance, the stent may be in the form of a counterwound and/or braided structure (e.g., one or more sections of the stoma stent may be formed of counterwound or braided material). Alternatively or additionally, the material of the stent may be elastically resilient and may cause the stent (or at least a portion thereof) to self-expand once a restraining force on the stent, holding the stent in an unexpanded or constrained or delivery configuration (such terms being used interchangeably herein without intent to limit), is released. The material of the stent may, in some embodiments, be a shape memory material or alloy, such as Nitinol. In some embodiments, additionally or alternatively, the stent may be shaped and configured to exert, and optionally formed of a material which generates, a radially outward or radially expansive force on the body opening (e.g., stoma wall, or abdominal wall) which may decrease the rate of stenosis of the body opening (e.g., stoma). The low constant force of the stent against the lumen wall in which the stent body is positioned assists in ensuring there are no major effects on the lumen wall, and inhibits or prevents the lumen diameter from constricting. By altering, adjusting, or selecting various parameters of the stent (e.g., wire thickness, wire count, coating configuration, etc.), the radial force values may be varied or selected to achieve higher or lower prescribed value as required by the anatomical location. Examples of radial forces exerted by commercially available stents generally used in internal orifices include (dimensions of stents given in terms of a saddle region lumen diameter×length, approximation of force measured at approximately 50% expansion of the stent): 6×8 mm=0.08 N/mm; 8×8 mm=0.09 N/mm; 15×10 mm=0.114 N/mm; 15×15 mm=0.120 N/mm; 20×10 mm=0.18 N/mm. In accordance with principles of the present disclosure, a stent used in a stoma contacts external skin surface and generally stronger or thicker muscle walls than contacted by stents implanted internally. Accordingly, it may be beneficial for a stoma stent formed in accordance with principles of the present disclosure to exert a greater force than typically exerted by internally-implanted stents, such as at least about 0.08 N/mm, and ranging (in increments of 0.01 N/mm) up to about 1.0 N/mm (±0.05 N/mm).

A stent in accordance with principles of the present disclosure may be formed of one or more interwoven stent filaments. As used herein, interwoven includes braided stent filaments, knitted stent filaments, and knotted stent filaments. Alternatively, the stent may alternatively be laser cut. A stent formed in accordance with principles of the present disclosure can be a combination of any of the above-mentioned stent types.

Suitable materials for the stent filaments include alloys such as Elgiloy™ and Nitinol, and polymers such as polyethylene terephthalate (PET). The stent filaments may be cored or composite fibers, e.g., having a Nitinol outer shell and a platinum core. Some examples of cored or composite fibers are disclosed in U.S. Pat. No. 7,101,392, titled Tubular Medical Endoprostheses, and issued to Heath on Sep. 5, 2006; and 6,527,802, titled Clad Composite Stent, and issued to Mayer on Mar. 4, 2003, each of which patent is incorporated herein by reference in its entirety for all purposes.

The overall shape of the stent may also be configured to reduce migration of the stent relative to the body opening or lumen in which the stent is deployed. Additionally, or alternatively, the overall shape of the stent may be configured to reduce the occurrence of retracted and prolapsed openings such as stomas. In some embodiments, a retention member, such as a flange, is provided on an end of the stent extending outwardly from the body opening/lumen in which the stent body is deployed. The retention member extends generally transverse to the longitudinal axis of the body lumen in which the stent body is deployed. The proximal retention member may be shaped and configured and positioned relative to the body opening or lumen to act as a retention mechanism, such as to prevent migration of the vessel wall of a stoma and/or otherwise to prevent stenosis, prolapse, or retraction of a stoma in which the stent is deployed. In some embodiments, the stent may have a retention member at each end. In the case of an opening such a stoma, the proximal retention member (at the proximal end of the stent) is positioned exterior to the internal body passage (e.g., vessel or intestine) in which the stent body is positioned, and a distal retention member (at a distal end of the stent) is positioned within the body and smaller in diameter (in a direction transverse to the longitudinal axis of the body lumen) than the proximal retention member. The proximal or exterior retention member may be in the form of a double-wall flange (e.g., formed from overlapping or folding of a portion of a substantially tubular stent wall upon radial expansion of the stent and generally concurrent axial shortening of the stent), or a single-wall flange. The proximal retention member may be shaped and configured to extend across a sufficient area around the opening in which the stent is deployed to hold the stent in place relative to the lumen in which the stent body is deployed. For instance, the proximal retention member may be shaped and configured to extend across a sufficient area of the exterior body wall surrounding a stoma in which the stent is deployed to anchor or otherwise hold the stent in place, thereby reducing the possibility of the stoma prolapsing or retracting. Radial force, exerted by the portion of the stent extending within the body lumen (typically, the force tangential to the force of the tissue wall of the opening in which the stent body is positioned, countering the forces of the tissue wall pushing inward tending to close the opening), along with the proximal retention member may retain the stent in place. The retention member may be considered to exert retention or retentive or holding forces opposing the force to remove the stent, such as when fully expanded and with little or no additional radial force exerted by the stent on the tissue in which the stent is positioned. Typically, the retentive force exerted by the retention member is tangential to the tissue wall surface (or at least includes a tangential component), countering a force exerted on the stent to pull the stent out of place, such as by deforming the retention member to pull it through the opening, generally without a radial force being applied. In accordance with principles of the present disclosure, a retention member with a higher retention or holding force than in typical stents may be desirable, such as in view of potential external forces (e.g., movement of the portion of the device caused by forces outside the body, or possible snags), and may be as high as in the range of approximately 10N. The retention or retentive or holding force of the retention member may be varied or altered or adjusted based on the flange size, braid angle, coating configuration and/or thickness, wire diameter, wire count, etc.

The proximal retention member may extend over any tissue which extends out from the body opening (e.g., an everted vessel wall extending outwardly from a stoma opening) and over an exterior body surface, and may protect such tissue as well. The retention member may provide sufficient protection to external body tissue to reduce or to remove the need for a shield device to be worn over the body opening to protect the body opening. As such, the proximal retention member of a stent formed in accordance with principles of the present disclosure is generally larger in diameter than retention members or flanges of prior art stents used in different anatomical sites and/or for different medical procedures (for example, currently commercially available 6×8 mm stents generally may have a retention member/flange diameter of about 14 mm, 8×8 mm stents generally may have a retention member/flange diameter of about 17 mm, and 20×10 mm stents generally may have a retention member/flange diameter of about 14 mm). For example, a 20 mm diameter stent (diameter of the main body region) may have a flange diameter of approximately 40 mm (±2 mm), and even up to 60 mm (±2 mm) (including values therebetween in increments of 1 mm), to aid in distributing retention member force over a less focal area around the body opening. It will be appreciated that a desired holding force against pull-out (e.g., up to about 10N of holding force) may be achieved through manipulation of other design elements (e.g., wire diameter, wire count, coating configuration and/or thickness, braid angle, etc.). Moreover, when one end of the stent extends out of a body opening and the other end of the stent remains in the body lumen in which the stent body is deployed, the retention member positioned at the body opening may be significantly greater than a retention member at the other end of the stent within the body lumen.

Various additional features may be provided on a retention member of a stent formed in accordance with principles of the present disclosure for adapting the stent for use in a particular body opening such as a stoma. For instance, the retention member may be formed to provide a retention force to prevent migration of the stent, and to reduce the likelihood of stenosis, prolapse, or retraction of a stoma. As such, a stent formed in accordance with principles of the present disclosure may have retention members or flanges with greater holding strength than provided in prior art stents. Additionally or alternatively, a retention member may be configured to permit cleaning of the body opening in which the stent is deployed, such as a stoma site. Additionally or alternatively, a stent may include a port or connector feature, such as on or near the retention member, permitting connection of the stent with an ostomy bag or other ostomy appliance, such as for collection of material emitted from a stoma in which the stent is deployed.

A simplified delivery system (compared with stent delivery systems used to deploy stents within body cavities or between body lumens or organs internal to the body) may be used to deploy a stent formed in accordance with principles of the present disclosure into a body opening such as an exterior body opening such as a stoma. In some embodiments, once a procedure forming a stoma has been completed and the vessel wall has been sutured to the skin surface (e.g., the exterior surface of the abdominal wall in the case of a colostomy), the medical professional can place the stent into the stoma. A stoma stent placed in a newly created stoma will help the healing process and ensure the lumen parameters of the stoma are maintained or set. Radially-outward forces of the stent may facilitate placement of the stent as well as the stoma (vessel wall extending through the body wall) to prevent migration of the stoma.

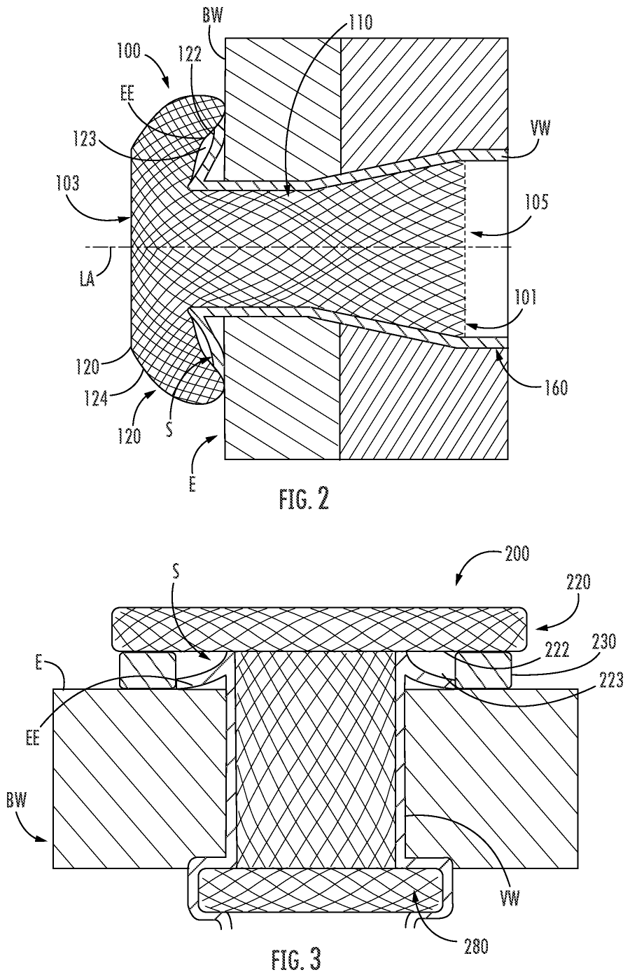

Turning now to the figures, various embodiments of various features of stents formed in accordance with principles of the present disclosure are illustrated in FIGS. 2, 3, 4A, 4B, and 5-10. One example of a stent 100 in accordance with principles of the present disclosure is illustrated schematically extending through a stoma S in FIGS. 1 and 2. It will be appreciated that the stent 100 illustrated in FIG. 2. is only one example of a stent which may be used in accordance with principles of the present disclosure, and other stents formed in accordance with principles of the present disclosure, such as described in further detail below, may be positioned as illustrated in FIGS. 1 and 2. As such, any references made to features of the stent 100 with reference to FIG. 1 is for the sake of convenience and without intent to limit the description of general principles to specific features of the stent 100 illustrated in FIG. 1. It will further be appreciated that although FIG. 1 illustrates a colostomy stoma, stents formed in accordance with principles of the present disclosure may be used in other stoma sites as well, or in other body openings or within other body lumens.

As may be appreciated with reference to FIG. 2, a stent 100 formed in accordance with principles of the present disclosure may be positioned with a distal end 101 within a body passage or body lumen (e.g., within the portion of the bowel extended to the stoma S) and a proximal end 103 extending outside the body passage or body lumen (e.g., in/over a stoma S along the exterior E of the body of the patient, such as against the skin of the patient). The stent 100 may extend through a body wall BW (such as an abdominal wall) to exit the body passage or body lumen (such terms being used interchangeably herein without intent to limit). A stent formed in accordance with principles of the present disclosure may include a tubular radially-expandable wall defining a stent body with a stent lumen or conduit or channel 105 (such terms being used interchangeably herein without intent to limit) therethrough to allow drainage therethrough (such as through the stoma S) as needed. Optionally, a valve may be provided within the channel 105, in any form known or heretofore known in the art for such uses, such as a one-way valve, e.g., butterfly or pinch, globe, plug valves, etc., such valve optionally being actuatable in conjunction with a device coupled to the stent (e.g., an ostomy appliance such as an ostomy bag).

The stent 100 may include a saddle region 110 configured to extend within a vessel wall VW within the body (such as illustrated in FIG. 2). The proximal end 103 of the stent 100 may be enlarged to form a retention member 120, such as in the form of a flange. In accordance with principles of the present disclosure, the retention member 120 is provided or formed on the proximal end 103 of the stent 100 to provide features which may be particularly beneficial for use of the stent 100 in various locations, such as a stoma. As described above, the retention member 120 may be shaped and configured to retain the stent 100 in place relative to an opening at the end of the body passage in which the stent body (e.g., a saddle region 110 of the stent body) is positioned (e.g., relative to a stoma S). For instance, the retention member 120 may be shaped and configured to be transverse to the body passage to inhibit or prevent migration of the stent 100 into (e.g., receding of) the body passage, thereby holding the stent 100 in the desired position. The retention member 120 may optionally include additional features configured specifically for use of the stent 100 in a particular anatomical site such as an ostomy stoma, such as described in further detail below. Various embodiments of configurations of retention members are illustrated in the accompanying drawings in stents with selected configurations of other features formed in accordance with various principles of the present disclosure. It will be appreciated that any of the retention member configurations described herein may be used with other stent features than the features illustrated in the embodiment in which a given embodiment of a retention member is illustrated. In general, the configuration of any particular feature described herein (e.g., features of a retention member) may be applied in conjunction with any of the other features of stents disclosed herein, such as in connection with other embodiments. In general, it will also be understood that the various embodiments of configuration of retention members may be used in stents for uses within openings or lumens, cavities, vessels, and the like, that are internal to the body.

In one embodiment, such as illustrated in FIG. 2, a stent 100 may be formed with a retention member 120 shaped and configured to extend across the opening in which the stent 100 is deployed. For instance, when positioned in a stoma, the retention member 120 covers the everted end EE of the vessel wall VW (in which the saddle region 110 is positioned) extended through the body wall BW to the exterior E of the body of the patient. It will be appreciated that the diameter of the retention member 120 may be selected based on the size of the opening through/from which the retention member 120 extends. In some embodiments, the retention member 120 has a diameter greater than the diameter of the body opening. As used with a stoma S, the retention member 120 diameter is greater than the diameter of the everted end EE of the vessel wall VW (since the stoma S and the everted end EE of the vessel wall VW are not necessarily circular, the diameter of either is to be understood herein as the greatest width across either), and may be as large as approximately 20 mm (±2 mm), or more, such as up to about 60 mm (±2 mm) (including values therebetween in increments of 1 mm), depending on the patient. In some embodiments, the retention member 120 is a radially extending flange shaped and configured to engage the body portion surrounding the opening in/through which the stent 100 is deployed. For instance, when deployed in a stoma S, the retention member 120 of the stent 100 may be shaped and configured to engage the exterior of the body wall BW to retain the stent 100 in place and to inhibit or to prevent retraction of the stent 100 and/or the stoma S into the body. The size of the proximal side 124 of the retention member 120 may be selected to distribute and dissipate external forces which may impact the stent 100, thereby reducing pressure at any given point. The distal end 101 of the stent 100 optionally may also be configured in a variety of configurations to enhance retention of the stoma stent 100 in place with respect to the body lumen in which it is deployed (e.g., within the stoma S and with respect to the body wall BW and vessel wall VW), as described in further detail below.

Since a stoma generally has exposed delicate tissue, it is desirable to exercise care with regard to the stoma. Generally, it would be desirable not to exert pressure on or abrade or otherwise contact the delicate tissue to a point the tissue may become injured or even necrose. Therefore, it may be desirable to permit some space between the body-facing distal side 122 of the retention member 120 and the everted end EE of the vessel wall VW. In some embodiments, the stent and the retention member are configured to form a gap between the proximal side of the retention member and the body tissue surrounding the opening from which the proximal end of the stent extends, such as the stoma. For example, the distal side 122 of the retention member 120 of the stent 100 illustrated in FIG. 2 may be angled with respect to the body wall BW and angled with respect to the longitudinal axis LA of the stent 100 to form a gap 123 between the body-facing distal side 122 of the retention member 120 and the body wall surrounding the opening from which the stent 100 extends, such as to provide space for an everted end EE of a vessel wall VW extending out of the opening. Such gap leaves the everted end EE of the vessel wall VW essentially untouched by the retention member 120 yet protected by the retention member 120, relieving and/or preventing any pressure or forces that the stoma might otherwise endure during daily use. Such gap 123 may also transfer any forces from the retention member 120 further from the opening from which the stent 100 extends, such as to the skin on the exterior E of the body of the patient surrounding the stoma S which the retention member 120 may contact (radially spaced from the stoma). The proximal side 124 of the retention member 120 may similarly be angled, such as to form a retention member 120 with a “mushroom” shape, as illustrated in FIG. 2. However, it will be appreciated that the proximal side 124 of the retention member 120 may have any other desired shape or configuration, not necessarily angled with respect to the longitudinal axis LA of the stent 100 to the same extent that the distal side 122 is angled. The periphery of the retention member 120 may be shaped to have a gently curved surface with a radius of curvature larger than in standard stents to present an atraumatic surface which may contact the body tissue surrounding the opening from which the stent 100 and retention member 120 extend.

In some embodiments, such as the stent 200 illustrated in FIG. 3, the retention member 220 is sufficiently sized to cover and extend beyond the periphery of the stoma S (as in the embodiment of FIG. 2), but the distal side 222 of the retention member 220 may not itself be shaped or configured to form a gap between the distal side 222 of the retention member 120 and the stoma S (e.g., the retention member 220 may be relatively flat or otherwise substantially follow the contour of the exterior E of the body of the patient at which the stoma S is formed). If it is desired to create a stoma gap 223 (such as for reasons as described above), the stent 200 may be provided with a spacer 230 (formed separately from the retention member 220 or incorporated with the retention member 220 such as a projection thereof) between the distal side 222 of the stent 200 and the exterior E of the body of the patient surrounding the stoma S to protect the stoma S (e.g., by being covered by the retention member 220) while not contacting the everted end EE of the vessel wall VW and/or the stoma S. In one embodiment, the spacer 230 may be in the form of a spacing ring adhered to the skin surrounding the stoma S so that the spacer 230 remains in place, yet is replaceable if needed or desired.

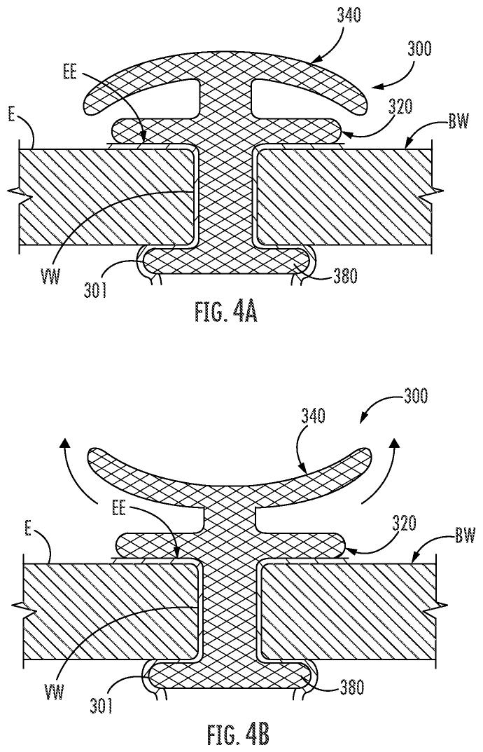

If desired, a retention member of a stent formed in accordance with principles of the present disclosure may have an additional external member (such as a flange) larger than the proximal, body-contacting retention member (which may function as a stabilizing flange), as in the stent 300 embodiment illustrated in FIGS. 4A and 4B. As may be appreciated, a covering member 340 may be sized and/or shaped and/or configured with respect to the primary retention member 320 to extend beyond the perimeter of the primary retention member 320, such as beyond the periphery of the everted end EE of the vessel wall VW. As such, the covering member 340 may act as a protective cap, isolating the stent deployment site from outside influence. The covering member 340 may be angled towards the exterior E of the body of the patient to form a convex shielding layer over the deployment site as well as the stent 300. Optionally, the covering member 340 can be flexible and formed to be lifted (such as to “flip” into a concave shape) away from the deployment site (generally with a force applied thereto when desired) to allow access to the site such as for assessment and hygiene considerations. The covering member 340 may have a biocompatible coating, such as formed of silicone, urethane elastomers, polymethyl methacrylate (PMMA), Chronoflex®, and the like, such that the wire construction is encapsulated in the coating and tissue ingrowth is inhibited or prevented.

A further embodiment of a stent 400, as illustrated in FIG. 5, could have an integrated connector 450 as part of the retention member 420 (extending therefrom and formed as an extension thereof or separately formed and coupled thereto) such that an irrigation or aspiration device or a collection receptacle or other ostomy appliance can be attached thereto and in communication with the channel 405 extending through the stent 400, as needed or desired. It will be appreciated that such connector 450 may be provided in any of the other stent embodiments disclosed herein or otherwise formed in accordance with principles of the present disclosure.

Returning to FIG. 2, the saddle region 110 of the stent 100 preferably extends distally into the body, as illustrated. As described briefly above, the distal end 101 of the stent 100 may be configured to enhance retention of the stent 100 in place within the body lumen in which the saddle region 110 is positioned, such as to prevent migration of the stent 100 and/or to prevent other undesired shifting of the body lumen or opening (e.g., prolapse of a stoma S). It will be appreciated that any of the stent distal end configurations described herein may be used with other stent features than the features illustrated in the embodiment in which a given embodiment of a distal end configuration is illustrated.

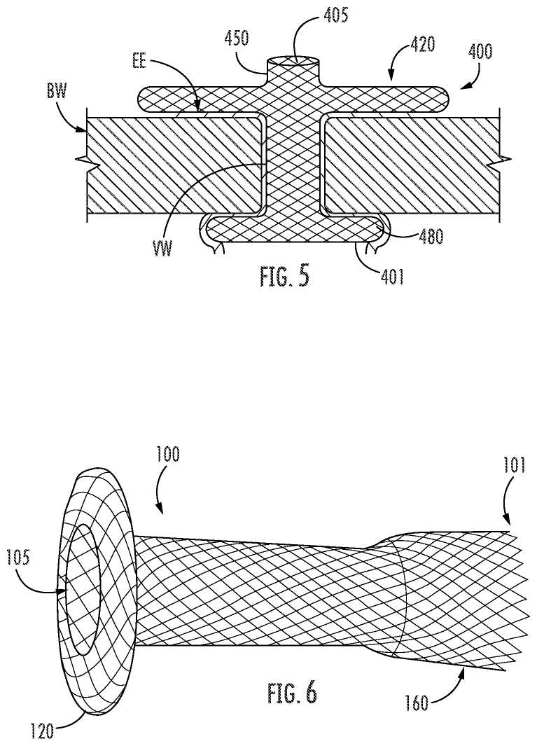

As illustrated in the embodiment of FIG. 2 (showing an example of a stent 100 extending through a stoma S) and FIG. 6 (showing a perspective view of a stent such as the stent 100 illustrated in FIG. 2), the distal end 101 of a stent 100 formed in accordance with principles of the present disclosure may be in the form of a flared section 160. The flared section 160 expands once deployed within the vessel wall VW of the lumen or vessel in which the stent 100 is deployed, and may have a degree of flare selected to reduce migration of the stent 100 relative to the vessel wall VW, yet may not apply much force against the opening through which the proximal end 103 of the stent 100 extends or the interior side of the body wall BW. If desired, radial forces applied by such flare may vary along the length of the flare (e.g., vary with the varying diameter of the flare), or a coating applied thereto may alter the radial forces of the flared section 160. In some embodiments, the flared section 160 may be formed of a different material or may be left at least partially uncoated, or otherwise, to further inhibit migration. Preferably, the flared section 160 has sufficient radial strength to inhibit internal stenosis of the vessel wall VW.

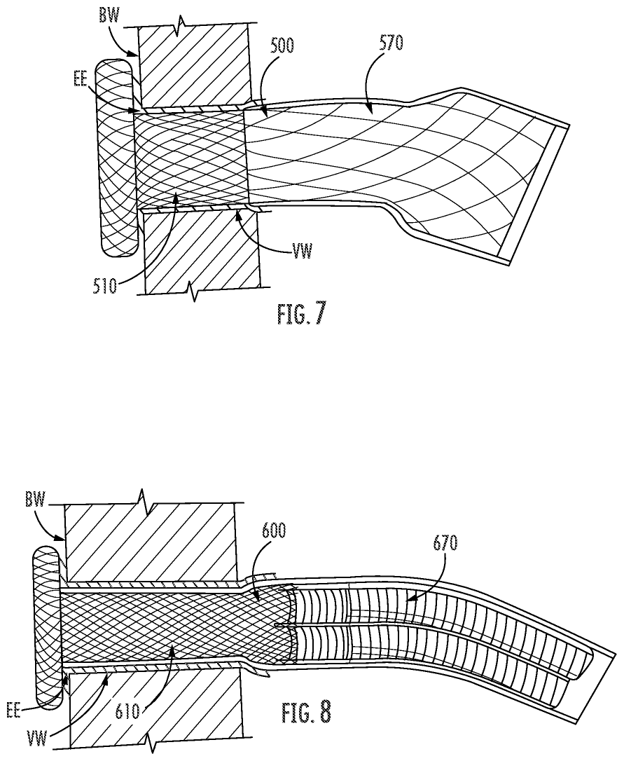

In some embodiments, a sleeve portion may extend distally from (e.g., be attached to or formed with) a stent, such as in the stent 500, 600 illustrated in FIGS. 7 and 8. A sleeve portion 570, 670 may extend from the saddle region 510, 610 of a stent 500, 600 distally within the vessel wall VW to reduce any irritation that might occur on the vessel wall VW. The sleeve portion 570, 670 may be a long conformable section trailing into the vessel wall VW, and may facilitate passage of materials out of the stoma. In some embodiments, the sleeve portion 570, 670 may have low radial and axial forces and be highly conformable. The sleeve portion 570 may be braided, as illustrated in FIG. 7, or the sleeve portion 670 may be knitted, as illustrated in FIG. 8.

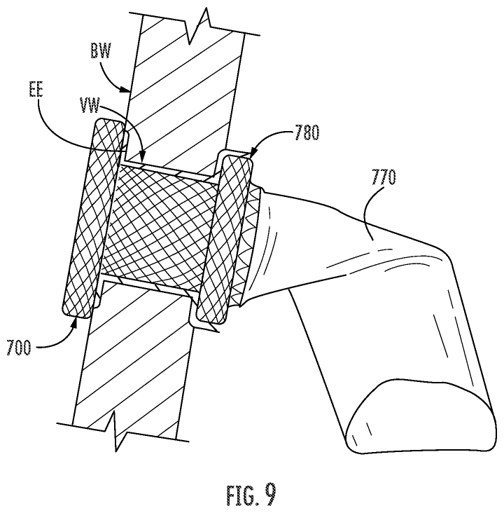

An additional internal retention member may be provided to facilitate retention of the stent such as by engaging the vessel wall VW, such as illustrated in the various examples of embodiments shown in FIGS. 3, 5, and 9. Such internal retention member may be formed or constructed similar to or different from the construction of the proximal retention member. A distal internal retention member, such as within the lumen in which the stent 100 is deployed, might be another design option to provide increased retention of the stent within the lumen and/or to reduce any residual risk of a vessel wall VW sliding in/out (retracting or prolapsing) through the body wall BW. In the embodiment of a stent 200 illustrated in FIG. 3 (described above with reference to the proximal/primary retention member 220), an internal retention member 280 is illustrated in the form of a radially-outwardly extending retention member (such as in the form of a flange) holding a section of the vessel wall VW in apposition to the interior side of the body wall BW. As shown in an embodiment of a stent 700 illustrated in FIG. 9, if desired, a sleeve portion 770, such as similar to either the sleeve portion 570 of FIG. 7, or the sleeve portion 670 of FIG. 8, may extend distally from an internal retention member 780.

It will be appreciated that an internal retention member 380 may be provided at the distal end 301 of the embodiment of a stent 300 illustrated in FIGS. 4A and 4B. Provision of an internal retention member 380 may enhance the retention of the stent 300 when the covering member 340 is lifted. Similarly, an internal retention member 480 may be provided at the distal end 301 of the embodiment of a stent 400 illustrated in FIG. 5 to enhance the retention of the stent 400 when a device is disconnected from the integrated connector 450.

In some embodiments, it may be desirable to cover or coat a portion of a stent formed in accordance with principles of the present disclosure with a polymeric coating such as to prevent leakage across the stent, and/or to inhibit or prevent tissue ingrowth, and/or to facilitate removal of the stent if desired or needed. In some embodiments, a polymeric material is applied to the stent in any suitable manner to form a polymeric cover, such, without limitation, dip coating or spray coating. Alternatively, a polymeric cover, such as a tubular cover, may be secured to a stent formed in accordance with principles of the present disclosure, such as by an interference fit, by sutures, or by any other suitable means of securement. Suitable materials for a polymeric cover or coating include silicone; styrene isoprene butadiene (SIBS); expanded polytetrafluoroethylene (ePTFE); polyurethane; rubbers; polyethylenes; polyvinylidene fluoride or polyvinylidene difluoride (PVDF); thermoplastic elastomers; and combinations thereof. The polymeric cover or coating may be made of a material that swells and/or coated with an agent that swells in situ. In some embodiments, the distal side (facing the everted vessel wall) and/or proximal side the retention member may be uncoated or partially uncoated to allow some tissue ingrowth, such as from the vessel wall VW that has been everted, such as to further inhibit migration (prolapse or retraction) of the tissue at the deployment site and/or to enhance retention of the retention member and stent in place relative to the deployment site to maintain the desired configuration and/or patency of the passage or opening or lumen in which the stent is deployed. Alternatively or additionally, the internal flare or internal retention member may be partially coated to allow some degree of tissue ingrowth, further stabilizing the stent position.

It is envisioned that a stent device formed in accordance with any of the above features in any suitable combination would be placed as one of the last steps of a procedure as a preemptive step to prevent complications. However, the device could also be placed in patients with one of the complications discussed above as a treatment option. The device would reverse the complication or prevent further progression of the complication. If the device is placed in time, the rates for more severe treatment procedures, such as relocation, could be reduced.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The following claims are hereby incorporated into this Detailed Description by this reference, with each claim standing on its own as a separate embodiment of the present disclosure. Reference signs in the claims are provided merely as a clarifying example and shall not be construed as limiting the scope of the claims in any way.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “the”, “first”, “second”, etc., do not preclude a plurality. Reference signs in the claims are provided merely as a clarifying example and shall not be construed as limiting the scope of the claims in any way.

What is claimed is:

1. A stent configured for deployment through a body opening and into a body lumen, said stent comprising:
a radially-expandable body portion movable between a delivery configuration, with a first diameter sized to pass into the body lumen, and an expanded configuration with a second diameter larger than the first diameter and sized to exert a radially expansive force on the body lumen wall; and
a retention member disposed on an end of said stent and configured to be deployed external to the body lumen and sized and shaped and configured to surround and cover the body opening to the body lumen and to retain said stent in place relative to the body lumen.

2. The stent of claim 1, wherein said retention member has a diameter selected to be larger than the diameter of a stoma in which said stent is to be placed to contact an exterior body wall surrounding a stoma in which said stoma device is placed.

3. The stent of claim 2, wherein said retention member distributes forces on said stent around the stoma to dissipate forces and to protect the stoma.

4. The stent of claim 1, wherein said stent further comprises a saddle region, said retention member extending radially-outwardly from said saddle region and shaped and configured to form a gap between a proximal side of the retention member and body tissue surrounding the opening.

5. The stent of claim 4, wherein said retention member has a distal side facing toward said saddle region and angled with respect to a longitudinal axis of the stent and toward said saddle region to form said gap.

6. The stent of claim 4, wherein said stent further includes a spacer positioned along a distal side of said retention member facing said saddle region to form said gap.

7. The stent of claim 1, further comprising a covering member proximal to said retention member and sized to extend beyond the perimeter of said retention member.

8. The stent of claim 7, wherein said covering member is flexible and liftable away from said retention member to expose the opening covered by said retention member.

9. The stent of claim 1, further comprising a connector extending from said retention member and configured for coupling with an ostomy appliance.

10. The stent of claim 1, further comprising an internal retention member configured to be positioned within the body lumen internal to the stoma.

11. A stoma device configured for deployment in a stoma, said stoma stent comprising:
a radially-expandable stent
having a retention member disposed on an end thereof;
wherein said retention member is configured to be placed external to the stoma and sized and shaped and further configured to cover the stoma and to adapt said stoma device for use in the stoma.

12. The stoma device of claim 11, wherein said retention member has a diameter selected to be larger than the diameter of the stoma in which said stoma device is to be deployed and is sized and shaped and configured to contact an exterior body wall surrounding a stoma in which said stoma device is deployed to distribute forces on said stoma device around the stoma to dissipate forces and to protect the stoma.

13. The stoma device of claim 11, wherein said stoma device further comprises a saddle region, said retention member extending radially-outwardly from said saddle region and shaped and configured to form a gap between a proximal side of said retention member and the stoma covered by said retention member.

14. The stoma device of claim 13, wherein said retention member has a distal side facing toward said saddle region and angled with respect to a longitudinal axis of the stent and toward said saddle region to form said gap.

15. The stoma device of claim 13, wherein said stoma device further includes a spacer positioned along a distal side of said retention member facing said saddle region to form said gap.

16. The stoma device of claim 11, further comprising a covering member proximal to said retention member and sized to extend beyond the perimeter of said retention member.

17. The stoma device of claim 16, wherein said covering member is flexible and liftable away from said retention member to expose the stoma covered by said retention member.

18. The stoma device of claim 11, further comprising a connector extending from said retention member and configured for coupling with an ostomy appliance.

19. A method of inhibiting migration of a stoma, said method comprising:
inserting a stoma device, having a radially-expandable saddle region and a retention member disposed on one end of the saddle region, into the stoma, the stoma device movable between an unexpanded configuration, with a compressed diameter sized to pass into the stoma, and an expanded configuration with an expanded diameter larger than the compressed diameter and sized to support the stoma; and
selecting the stoma device to exert a radially-expansive force on the stoma to retain the stoma device in place relative to the stoma to prevent migration of both the stoma device and the stoma.

20. The method of claim 19, wherein the stoma device further comprises a retention member configured to be placed external to the stoma, said method further comprising selecting a stoma device with a retention member sized and shaped and configured to cover the stoma site, and covering the stoma with the retention member upon placement of the stoma device within the stoma.
